(19) United States
(12) Patent Application Publication
(10) Pub. No.: US 2019/0117899 A1
(43) Pub. Date: Apr. 25, 2019

(54) ACCESSORY DEVICE WITH ZERO POSITION ADJUSTMENT FEATURE

(71) Applicant: Novo Nordisk A/S, Bagsvaerd (DK)

(72) Inventors: Nikolai Byskov, Noerrebro (DK); Michael Ejstrup Hansen, Morud (DK); Per Einar Pontus Holm, Tygelsjoe (SE)

(21) Appl. No.: 16/095,100

(22) PCT Filed: Apr. 27, 2017
(86) PCT No.: PCT/EP2017/060039

§ 371 Date: Oct. 19, 2018

(30) Foreign Application Priority Data
Apr. 28, 2016 (EP) 16167466.8

(51) Int. Cl. A61M 5/315; A61M 5/24

(52) U.S. Cl. CPC A61M 5/31553; A61M 5/24; A61M 5/31556; A61M 2005/3126; A61M 5/31583; A61M 5/31541; A61M 5/31528

(57) ABSTRACT

An add-on device configured to be releasably attached to a drug delivery device comprising a housing with a reference marker and a scale drum with a zero identifier and an initial marker. The add-on device comprises a memory and a camera adapted to capture an image of the housing reference marker and the initial marker when the scale drum is in a (near) initial position. The add-on device is adapted to perform a reference offset value determination, comprising: capture an image, perform an image analysis to determine if the captured image comprises the zero identifier, if so, determine a reference offset value based on the distance between the housing reference marker and the initial marker, the reference off-set value representing the initial rotational position, and if no reference offset value is stored in the memory or if the determined reference offset value corresponds to a smaller set dose amount than a currently stored value, store in the memory the determined reference offset value.

The present invention generally relates to medical devices for which the generation, collecting and storing of data are relevant. In specific embodiments the invention relates to devices and systems for capturing drug delivery dose data in a reliable and efficient way.

### BACKGROUND OF THE INVENTION

In the disclosure of the present invention reference is mostly made to drug delivery devices comprising a threaded piston rod driven by a rotating drive member, such devices being used e.g. in the treatment of diabetes by delivery of insulin, however, this is only an exemplary use of the present invention.

Drug Injection devices have greatly improved the lives of patients who must self-administer drugs and biological agents. Drug Injection devices may take many forms, including simple disposable devices that are little more than an ampoule with an injection means or they may be durable devices adapted to be used with prefilled cartridges. Regardless of their form and type, they have proven to be great aids in assisting patients to self-administer injectable drugs and biological agents. They also greatly assist care givers in administering injectable medicines to those incapable of performing self-injections.

Performing the necessary insulin injection at the right time and in the right size is essential for managing diabetes, i.e. compliance with the specified insulin regimen is important. In order to make it possible for medical personnel to determine the effectiveness of a prescribed dosage pattern, diabetes patients are encouraged to keep a log of the size and time of each injection. However, such logs are normally kept in handwritten notebooks, and the logged information may not be easily uploaded to a computer for data processing. Furthermore, as only events, which are noted by the patient, are logged, the note book system requires that the patient remembers to log each injection, if the logged information is to have any value in the treatment of the patient's disease. A missing or erroneous record in the log results in a misleading picture of the injection history and thus a misleading basis for the medical personnel's decision making with respect to future medication. Accordingly, it may be desirable to automate the logging of injection information from medication delivery systems.

Though some injection devices integrate this monitoring/acquisition mechanism into the device itself, e.g. as disclosed in US 2009/0318865 and WO 2010/052275, most devices of today are without it. The most widely used devices are purely mechanical devices being either durable or prefilled. The latter devices are to be discarded after being emptied and so inexpensive that it is not cost-effective to build-in electronic data acquisition functionality in the device it-self. Addressing this problem a number of solutions have been proposed which would help a user to generate, collect and distribute data indicative of the use of a given medical device.

For example, WO 2013/120776 and WO 2015/110520 describe an electronic supplementary device (or “add-on module”) adapted to be releasably attached to a drug delivery device of the pen type. The device includes a camera and is configured to perform optical character recognition (OCR) on captured images from a rotating scale drum visible through a dosage window on the drug delivery device, thereby to determine a dose of medicament that has been dialled into the drug delivery device. In WO 2015/110520 the centre line of the optical sensor's field of view is used in the OCR process. A further external device for a pen device is shown in WO 2014/161952. As any given drug delivery device is manufactured with tolerances for each component also scale drum dose size indication accuracy will potentially vary for each device, e.g. for any given set dose the corresponding scale drum indicia, e.g. a line marking, may not be perfectly aligned with the housing pointer structure, this potentially resulting in inaccuracies when determining scale drum position and thus an incorrect determination of an expelled dose size.

Having regard to the above, it is an object of the present invention to provide devices and methods allowing reliable and cost-effective operation of a drug delivery assembly comprising a user-mountable logging module.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention an add-on device configured to be releasably attached to a drug delivery device is provided, the drug delivery device comprising a drug reservoir or a compartment for receiving a drug reservoir, drug expelling means comprising a dose setting member allowing a user to set a dose amount of drug to be expelled, an indicator member adapted to rotate relative to the housing during dose setting and dose expelling corresponding to an axis of rotation, the amount of rotation corresponding to a set dose respectively the amount of drug remaining to be expelled from a reservoir by the expelling means, the indicator member having an initial rotational position corresponding to no dose amount being set, a housing comprising an opening allowing a user to observe a portion of the indicator member, the opening being surrounded by a housing edge formed by the housing, the housing comprising a housing reference marker. A pattern is arranged circumferentially or helically on the indicator member and comprises a plurality of indicia, the currently observable indicia indicating to a user the size of a currently set dose amount of drug to be expelled, and an initial pattern portion observable by the user when the indicator member is positioned in the initial rotational position, the initial pattern portion comprising a pattern reference marker. The add-on device is adapted to determine, when mounted to a drug delivery device housing, an amount of rotation of the indicator member relative to the housing. The add-on device comprises mounting means adapted to releasably mount the add-on device to the drug delivery device in a predetermined position and orientation, a memory, and capturing means. The capturing means is adapted to capture an image of at least a portion of the initial pattern portion including the pattern reference marker, as well as the housing reference marker. The add-on device further comprises a processor adapted to perform an image analysis to identify the initial pattern portion, and adapted to determine an amount of rotation of the indicator member relative to the housing based on input from the capturing means. The add-on device is adapted to perform a reference offset value determination, comprising the steps of: capturing an image, performing an image analysis to determine if the captured image comprises the initial pattern portion, if the captured image comprises the initial pattern portion, determining a reference offset value based on the distance between the housing reference marker and the pattern reference marker, the reference off-set value representing the initial rotational position, and if no reference offset value is stored in the memory or if the determined reference offset value corresponds to a smaller set dose amount than a currently stored value, storing or replacing in the memory the determined reference offset value.

By this arrangement the stored reference offset value is a dynamic value which may be updated and overwritten in an automated and efficient way, the currently stored value thereby representing the “true” initial position of the scale drum. Correspondingly, an add-on device is provided which in a user friendly way is designed to adapt to potential manufacturing variations in the drug delivery device to which the add-on device is to be mounted.

In an exemplary embodiment the housing reference marker is formed by a portion of the housing edge. Alternatively, the housing reference marker could be a structure provided for the specific purpose of being a marker in the context of the present invention. For example, a marker symbol formed in the housing material could be provided in the vicinity of the housing edge. The marker symbol could be formed in the same material as the housing or a different material and/or colour in a 2K moulding process, e.g. to improve contrast. As a further alternative a marking may be applied to the housing surface in the vicinity of the housing edge after moulding of the housing, e.g. by printing. The housing may be provided with a pointer structure and the plurality of indicia may be associated with a plurality of dose size markers, the pattern reference marker being formed by a dose size marker. The pattern reference marker may be formed by the dose size marker for a set dose amount of zero which may be different from the remaining dose size markers.

In the memory a reference representation of at least a portion of the initial pattern portion may be stored, the processor being adapted to perform an image analysis comparing a captured image with the stored reference representation of at least a portion of the initial pattern portion.

In an exemplary embodiment the add-on device is adapted to be mounted on a drug delivery device in which the pattern arranged on the indicator member forms a dosing pattern comprising a plurality of pattern portions, each pattern portion being arranged corresponding to a position on the indicator member. Correspondingly, a reference representation of the dosing pattern is stored in the add-on device memory, each part of the stored dosing pattern being correlated with a rotational position of the indicator member, and the processor is adapted to perform a best-match analysis between a captured image and the stored reference representation to thereby determine the rotational position of the indicator member.

For the given drug delivery device a nominal reference offset value is defined as the distance between the housing reference marker and the pattern reference marker in its nominal initial position. The nominal reference offset value may be stored in the add-on device which may be adapted to determine an offset value (OV) as the difference between the nominal reference offset value (ROV-nom) and a determined reference offset value (ROV). Before the best-match analysis is performed, the rotational correlation between a captured image and the stored reference representation may be adjusted corresponding to the determined reference offset value. In this way the determined reference offset value can be used to determine the rotational position of the indicator member in reliable and efficient way.

In an exemplary embodiment the capturing means is arranged to capture an image with a given distortion, e.g. due to the angular orientation between the camera and the scale drum and/or the influence of any optical elements arranged in front of the camera, the reference representation being processed before storage to create a distorted representation matching the images actually captured. Alternatively, the capturing means is arranged to capture an image with a given distortion, the captured image being processed before the best-match analysis to create an image matching the stored reference representation.

In an exemplary embodiment an expelled dose amount is determined based on a first feature captured from a first image of the indicator member and a second feature captured from a second image of the indicator member, the first image being captured when a dose amount has been set and the second image being captured when a dose amount has been expelled. The first feature may be used to determine a first position of the indicator member, and the second feature may be used to determine a second position of the indicator member, the positional difference between the first and second positions being indicative of an expelled amount of drug.

The processor may be adapted to store in the memory data corresponding to one or more expelled dose amounts. The stored data corresponding to one or more expelled dose amounts may be updated in case a stored reference offset value is replaced with a new value.

The above-described add-on devices may be provided in combination with a drug delivery device as also described above.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof. In the description of exemplary embodiments reference will be made to the use of insulin, however, the described module could also be used to create logs for other types of drug, e.g. growth hormone.

### BRIEF DESCRIPTION OF THE DRAWINGS

In the following embodiments of the invention will be described with reference to the drawings, wherein

FIG. 1A shows a pen device,

FIG. 1B shows the pen device of FIG. 1A with the pen cap removed,

FIG. 2 shows in an exploded view the components of the pen device of FIG. 1A,

FIGS. 3A and 3B show in sectional views an expelling mechanism in two states,

FIG. 4 shows a schematic representation of an add-on device,

FIG. 5 shows an add-on device mounted on the housing of a drug delivery device,

FIGS. 6A and 6B show a scale drum in initial positions,

FIG. 7 shows a scale drum reference representation,

FIG. 8 shows an image capture from a scale drum,

FIG. 9 shows cross correlation of the FIG. 8 image portion to the reference representation, and

FIG. 10 shows a matched portion of the reference representation.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

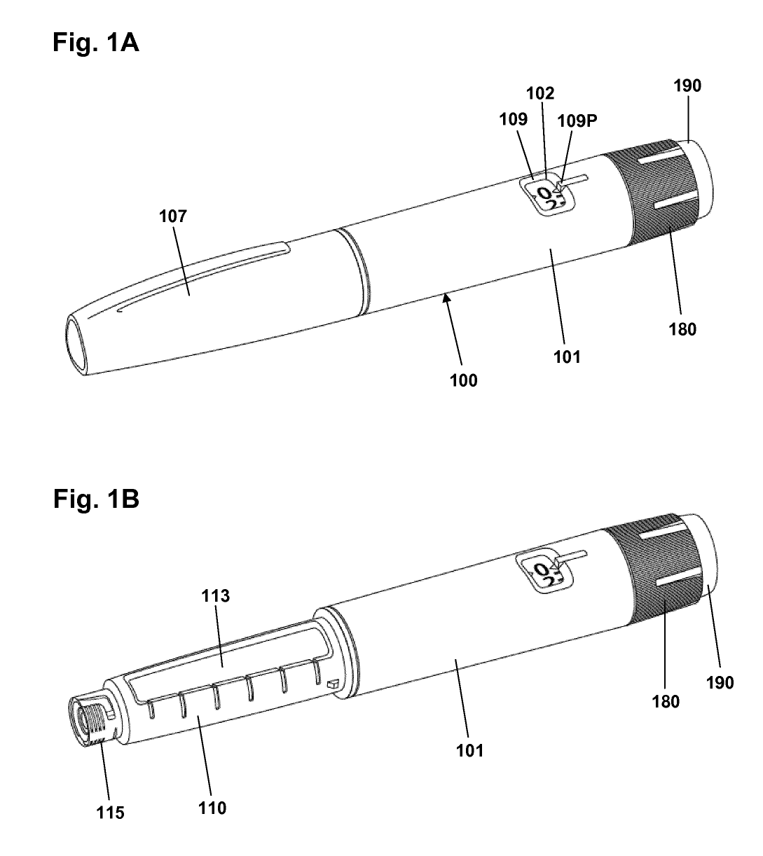

Before turning to embodiments of the present invention per se, an example of a prefilled drug delivery will be described, such a device providing the basis for the exemplary embodiments of the present invention. Although the pen-formed drug delivery device 100 shown in FIGS. 1-3 may represent a “generic” drug delivery device, the actually shown device is a FlexTouch® prefilled drug delivery pen as manufactured and sold by Novo Nordisk A/S, Bagsℏvrd, Denmark.

The pen device 100 comprises a cap part 107 and a main part having a proximal body or drive assembly portion with a housing 101 in which a drug expelling mechanism is arranged or integrated, and a distal cartridge holder portion in which a drug-filled transparent cartridge 113 with a distal needle-penetrable septum is arranged and retained in place by a non-removable cartridge holder attached to the proximal portion, the cartridge holder having openings allowing a portion of the cartridge to be inspected as well as distal coupling means 115 allowing a needle assembly to be releasably mounted. The cartridge is provided with a piston driven by a piston rod forming part of the expelling mechanism and may for example contain an insulin, GLP-1 or growth hormone formulation. A proximal-most rotatable dose setting member 180 serves to manually set a desired dose of drug shown in display window 102 and which can then be expelled when the button 190 is actuated. The window is surrounded by a chamfered edge portion 109 and a dose pointer 109P. Depending on the type of expelling mechanism embodied in the drug delivery device, the expelling mechanism may comprise a spring as in the shown embodiment which is strained during dose setting and then released to drive the piston rod when the release button is actuated. Alternatively the expelling mechanism may be fully manual in which case the dose member and the actuation button moves proximally during dose setting corresponding to the set dose size, and then is moved distally by the user to expel the set dose, e.g. as in a FlexPen® manufactured and sold by Novo Nordisk A/S.

Although FIG. 1 shows a drug delivery device of the prefilled type, i.e. it is supplied with a pre-mounted cartridge and is to be discarded when the cartridge has been emptied, in alternative embodiments the drug delivery device may be designed to allow a loaded cartridge to be replaced, e.g. in the form of a “rear-loaded” drug delivery device in which the cartridge holder is adapted to be removed from the device main portion, or alternatively in the form of a “front-loaded” device in which a cartridge is inserted through a distal opening in the cartridge holder which is non-removable attached to the main part of the device.

As the invention relates to electronic circuitry adapted to interact with a drug delivery device, an exemplary embodiment of such a device will be described for better understanding of the invention.

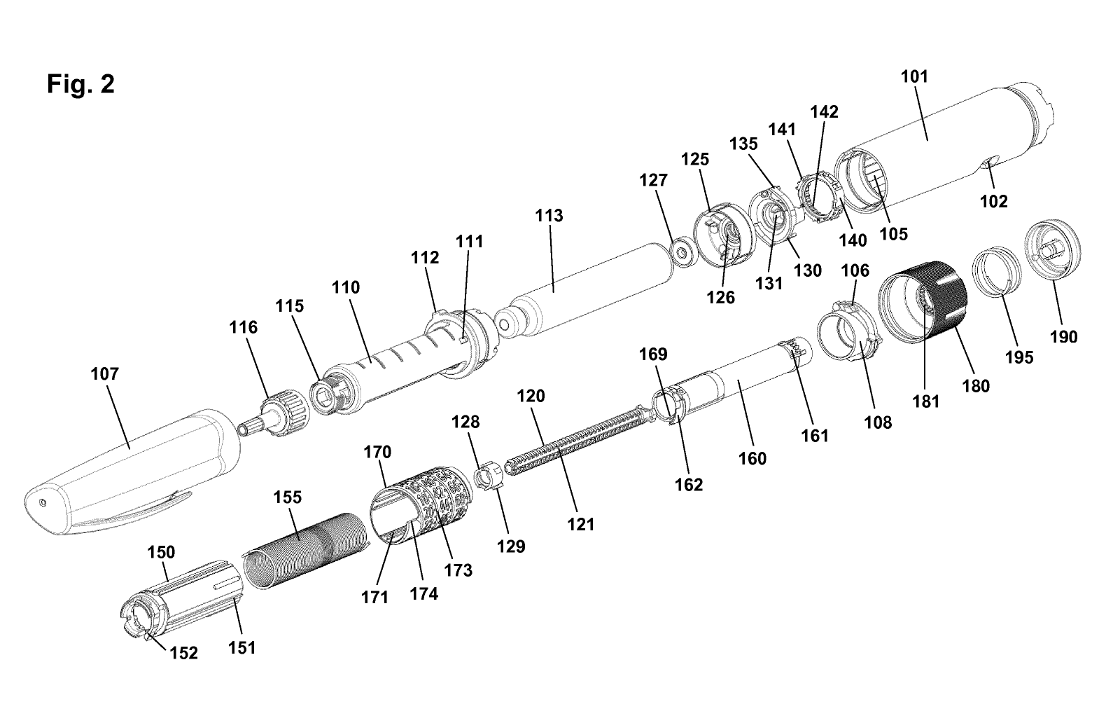

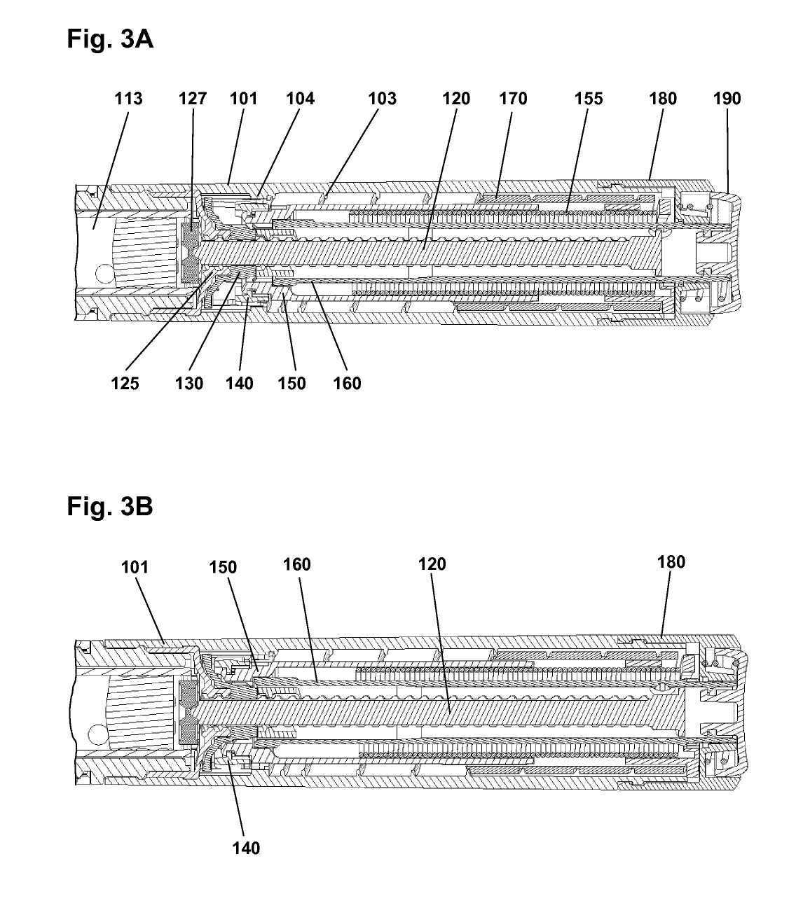

FIG. 2 shows an exploded view of the pen-formed drug delivery device 100 shown in FIG. 1. More specifically, the pen comprises a tubular housing 101 with a window opening 102 and onto which a cartridge holder 110 is fixedly mounted, a drug-filled cartridge 113 being arranged in the cartridge holder. The cartridge holder is provided with distal coupling means 115 allowing a needle assembly 116 to be releasable mounted, proximal coupling means in the form of two opposed protrusions 111 allowing a cap 107 to be releasable mounted covering the cartridge holder and a mounted needle assembly, as well as a protrusion 112 preventing the pen from rolling on e.g. a table top. In the housing distal end a nut element 125 is fixedly mounted, the nut element comprising a central threaded bore 126, and in the housing proximal end a spring base member 108 with a central opening is fixedly mounted. A drive system comprises a threaded piston rod 120 having two opposed longitudinal grooves and being received in the nut element threaded bore, a ring-formed piston rod drive element 130 rotationally arranged in the housing, and a ring-formed clutch element 140 which is in rotational engagement with the drive element (see below), the engagement allowing axial movement of the clutch element. The clutch element is provided with outer spline elements 141 adapted to engage corresponding splines 104 (see FIG. 3B) on the housing inner surface, this allowing the clutch element to be moved between a rotationally locked proximal position, in which the splines are in engagement, and a rotationally free distal position in which the splines are out of engagement. As just mentioned, in both positions the clutch element is rotationally locked to the drive element. The drive element comprises a central bore with two opposed protrusions 131 in engagement with the grooves on the piston rod whereby rotation of the drive element results in rotation and thereby distal axial movement of the piston rod due to the threaded engagement between the piston rod and the nut element. The drive element further comprises a pair of opposed circumferentially extending flexible ratchet arms 135 adapted to engage corresponding ratchet teeth 105 arranged on the housing inner surface. The drive element and the clutch element comprise cooperating coupling structures rotationally locking them together but allowing the clutch element to be moved axially, this allowing the clutch element to be moved axially to its distal position in which it is allowed to rotate, thereby transmitting rotational movement from the dial system (see below) to the drive system. The interaction between the clutch element, the drive element and the housing will be shown and described in greater detail with reference to FIGS. 3A and 3B.

On the piston rod an end-of-content (EOC) member 128 is threadedly mounted and on the distal end a washer 127 is rotationally mounted. The EOC member comprises a pair of opposed radial projections 129 for engagement with the reset tube (see below).

The dial system comprises a ratchet tube 150, a reset tube 160, a scale drum 170 with an outer helically arranged pattern forming a row of dose indicia, a user-operated dial member 180 for setting a dose of drug to be expelled, a release button 190 and a torque spring 155 (see FIG. 3). The reset tube is mounted axially locked inside the ratchet tube but is allowed to rotate a few degrees (see below). The reset tube comprises on its inner surface two opposed longitudinal grooves 169 adapted to engage the radial projections 129 of the EOC member, whereby the EOC can be rotated by the reset tube but is allowed to move axially. The clutch element is mounted axially locked on the outer distal end portion of the ratchet tube 150, this providing that the ratchet tube can be moved axially in and out of rotational engagement with the housing via the clutch element. The dial member 180 is mounted axially locked but rotationally free on the housing proximal end, the dial ring being under normal operation rotationally locked to the reset tube (see below), whereby rotation of dial ring results in a corresponding rotation of the reset tube and thereby the ratchet tube. The release button 190 is axially locked to the reset tube but is free to rotate. A return spring 195 provides a proximally directed force on the button and the thereto mounted reset tube. The scale drum 170 is arranged in the circumferential space between the ratchet tube and the housing, the drum being rotationally locked to the ratchet tube via cooperating longitudinal splines 151, 171 and being in rotational threaded engagement with the inner surface of the housing via cooperating thread structures 103, 173, whereby the row of numerals passes the window opening 102 in the housing when the drum is rotated relative to the housing by the ratchet tube. The torque spring is arranged in the circumferential space between the ratchet tube and the reset tube and is at its proximal end secured to the spring base member 108 and at its distal end to the ratchet tube, whereby the spring is strained when the ratchet tube is rotated relative to the housing by rotation of the dial member. A ratchet mechanism with a flexible ratchet arm 152 is provided between the ratchet tube and the clutch element, the latter being provided with an inner circumferential teeth structures 142, each tooth providing a ratchet stop such that the ratchet tube is held in the position to which it is rotated by a user via the reset tube when a dose is set. In order to allow a set dose to be reduced a ratchet release mechanism 162 is provided on the reset tube and acting on the ratchet tube, this allowing a set dose to be reduced by one or more ratchet increments by turning the dial member in the opposite direction, the release mechanism being actuated when the reset tube is rotated the above-described few degrees relative to the ratchet tube.

Having described the different components of the expelling mechanism and their functional relationship, operation of the mechanism will be described next with reference mainly to FIGS. 3A and 3B.

The pen mechanism can be considered as two interacting systems, a dose system and a dial system, this as described above. During dose setting the dial mechanism rotates and the torsion spring is loaded. The dose mechanism is locked to the housing and cannot move. When the push button is pushed down, the dose mechanism is released from the housing and due to the engagement to the dial system the torsion spring will now rotate back the dial system to the starting point and rotate the dose system along with it.

The central part of the dose mechanism is the piston rod 120, the actual displacement of the plunger being performed by the piston rod. During dose delivery, the piston rod is rotated by the drive element 130 and due to the threaded interaction with the nut element 125 which is fixed to the housing, the piston rod moves forward in the distal direction. Between the rubber piston and the piston rod, the piston washer 127 is placed which serves as an axial bearing for the rotating piston rod and evens out the pressure on the rubber piston. As the piston rod has a non-circular cross section where the piston rod drive element engages with the piston rod, the drive element is locked rotationally to the piston rod, but free to move along the piston rod axis. Consequently, rotation of the drive element results in a linear forwards movement of the piston. The drive element is provided with small ratchet arms 134 which prevent the drive element from rotating clockwise (seen from the push button end). Due to the engagement with the drive element, the piston rod can thus only move forwards. During dose delivery, the drive element rotates anti-clockwise and the ratchet arms 135 provide the user with small clicks due to the engagement with the ratchet teeth 105, e.g. one click per unit of insulin expelled.

Turning to the dial system, the dose is set and reset by turning the dial member 180. When turning the dial, the reset tube 160, the EOC member 128, the ratchet tube 150 and the scale drum 170 all turn with it. As the ratchet tube is connected to the distal end of the torque spring 155, the spring is loaded. During dose setting, the arm 152 of the ratchet performs a dial click for each unit dialled due to the interaction with the inner teeth structure 142 of the clutch element. In the shown embodiment the clutch element is provided with 24 ratchet stops providing 24 clicks (increments) for a full 360 degrees rotation relative to the housing. The spring is preloaded during assembly which enables the mechanism to deliver both small and large doses within an acceptable speed interval. As the scale drum is rotationally engaged with the ratchet tube, but movable in the axial direction and the scale drum is in threaded engagement with the housing, the scale drum will move in a helical pattern when the dial system is turned, the number corresponding to the set dose being shown in the housing window 102.

The ratchet 152, 142 between the ratchet tube and the clutch element 140 prevents the spring from turning back the parts. During resetting, the reset tube moves the ratchet arm 152, thereby releasing the ratchet click by click, one click corresponding to one unit IU of insulin in the described embodiment. More specifically, when the dial member is turned clockwise, the reset tube simply rotates the ratchet tube allowing the arm of the ratchet to freely interact with the teeth structures 142 in the clutch element. When the dial member is turned counter-clockwise, the reset tube interacts directly with the ratchet click arm forcing the click arm towards the centre of the pen away from the teeth in the clutch, thus allowing the click arm on the ratchet to move “one click” backwards due to torque caused by the loaded spring.

To deliver a set dose, the push button 190 is pushed in the distal direction by the user as shown in FIG. 3B. The reset tube 160 decouples from the dial member and subsequently the clutch element 140 disengages the housing splines 104. Now the dial mechanism returns to “zero” together with the drive element 130, this leading to a dose of drug being expelled. It is possible to stop and start a dose at any time by releasing or pushing the push button at any time during drug delivery. A set dose of less than 5 IU normally cannot be paused, since the rubber piston is compressed very quickly leading to a compression of the rubber piston and subsequently delivery of insulin when the piston returns to the original dimensions. This said, a larger dose can be paused with only a few IU left to be expelled, e.g. as little as 1 IU.

The EOC feature prevents the user from setting a larger dose than left in the cartridge. The EOC member 128 is rotationally locked to the reset tube, which makes the EOC member rotate during dose setting, resetting and dose delivery, during which it can be moved axially back and forth following the thread of the piston rod. When it reaches the proximal end of the piston rod a stop is provided, this preventing all the connected parts, including the dial member, from being rotated further in the dose setting direction, i.e. the now set dose corresponds to the remaining drug content in the cartridge.

The scale drum 170 is provided with a distal stop surface 174 adapted to engage a corresponding stop surface on the housing inner surface, this providing a maximum dose stop for the scale drum preventing all the connected parts, including the dial member, from being rotated further in the dose setting direction. In the shown embodiment the maximum dose is set to 80 IU. Correspondingly, the scale drum is provided with a proximal stop surface adapted to engage a corresponding stop surface on the spring base member, this preventing all the connected parts, including the dial member, from being rotated further in the dose expelling direction, thereby providing a “zero” stop for the entire expelling mechanism.

To prevent accidental over-dosage in case something should fail in the dialling mechanism allowing the scale drum to move beyond its zero-position, the EOC member serves to provide a security system. More specifically, in an initial state with a full cartridge the EOC member is positioned in a distal-most axial position in contact with the drive element. After a given dose has been expelled the EOC member will again be positioned in contact with the drive element. Correspondingly, the EOC member will lock against the drive element in case the mechanism tries to deliver a dose beyond the zero-position. Due to tolerances and flexibility of the different parts of the mechanism the EOC will travel a short distance allowing a small “over dose” of drug to be expelled, e.g. 3-5 IU of insulin.

The expelling mechanism further comprises an end-of-dose (EOD) click feature providing a distinct feedback at the end of an expelled dose informing the user that the full amount of drug has been expelled. More specifically, the EOD function is made by the interaction between the spring base and the scale drum. When the scale drum returns to zero, a small click arm 106 on the spring base is forced backwards by the progressing scale drum. Just before “zero” the arm is released and the arm hits a countersunk surface on the scale drum.

The shown mechanism is further provided with a torque limiter in order to protect the mechanism from overload applied by the user via the dial member. This feature is provided by the interface between the dial member and the reset tube which as described above are rotationally locked to each other. More specifically, the dial member is provided with a circumferential inner teeth structure 181 engaging a number of corresponding teeth arranged on a flexible carrier portion 161 of the reset tube. The reset tube teeth are designed to transmit a torque of a given specified maximum size, e.g. 150-300 Nmm, above which the flexible carrier portion and the teeth will bend inwards and make the dial member turn without rotating the rest of the dial mechanism. Thus, the mechanism inside the pen cannot be stressed at a higher load than the torque limiter transmits through the teeth.

Having described the working principles of a mechanical drug delivery device, embodiments of the present invention will be described.

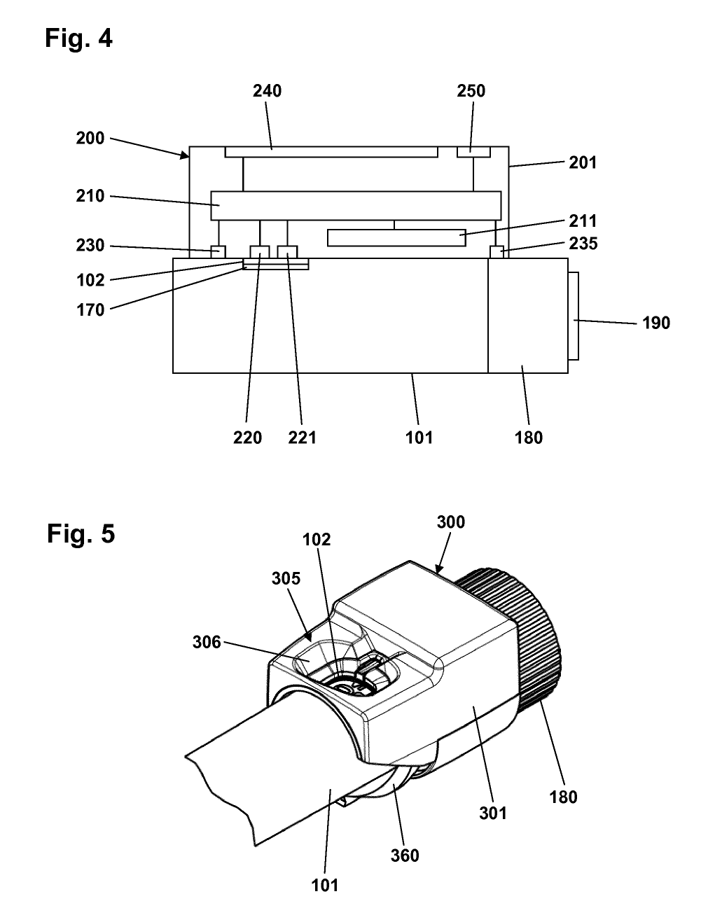

FIG. 4 shows a schematic representation of an add-on device 200 in a state where it has been mounted on the housing 101 of a drug delivery device 100 of the above-described pen type. The add-on device is adapted to determine the amount of drug expelled from the drug delivery device during an expelling event, i.e. the subcutaneous injection of a dose of drug. In the shown embodiment determination of an expelled dose of drug is based on determination of scale drum position at the beginning and at the end of the expelling event. To determine the rotational position of the scale drum the dose numerals as seen in the display window 102 may be captured and used, this allowing an unmodified pen device to be used. Actual determination of scale drum position may be performed using e.g. template matching (see below) or optical character recognition (OCR). Alternatively a dedicated code pattern may be provided on the scale drum as disclosed in e.g. WO 2013/004843.

The add-on device comprises a housing 201 in which is arranged electronic circuitry 210 powered by an energy source 211. The electronic circuitry is connected to and interacts with a light source 220 adapted to illuminate at least a portion of the scale drum 170 seen in the window 102, an image capture device (camera) 221 adapted to capture image data from the scale drum, a mounting switch 230 adapted to engage the pen housing 101, a display 240 and user input means in the form of one or more buttons 250. In the shown embodiment a further activity switch 235 adapted to engage the dose setting member 180 is provided. Alternatively or in addition an acoustic sensor may be provided to detect specific sounds generated by the expelling mechanism during dose setting and dose expelling. The electronic circuitry 210 will typically comprise controller means, e.g. in the form of a generic microprocessor or an ASIC, ROM and RAM memory providing storage for imbedded program code and data, a display controller and a wireless transmitter/receiver.

The add-on device further comprises mounting means (not shown) adapted to releasably mount and securely hold and position the add-on device on the pen housing. For the shown embodiment the add-on device covers the display window for which reason the current dose size shown in the display window has to be captured and displayed on the electronic display 240. Alternatively, the add-on device may be designed to allow the user to view the display window.

The coupling means may be in the form of e.g. a bore allowing the add-on device to slide in place on the pen body, flexible gripping structures allowing the add-on device to be mounted in a perpendicular direction, locking means that will snap in place when the add-on device is mounted on the pen body, or locking means which has to be operated by the user, e.g. a hinged latch member or a sliding member.

As scale drum position and thus dose size determination is based on image capturing and subsequent processing of the captured image data, it is important that the add-on device is correctly positioned in its intended operational position on the drug delivery device. Thus, in order to securely hold and position the add-on device on the pen housing the add-on device may be provided with positioning means adapted to engage a corresponding positioning structure on the pen body. The positioning structure may be in the form of an existing structure provided for a different purpose, e.g. the window opening, or a specific mounting structure, e.g. one or more indents provided on the pen body. In addition to the above-described coupling and positioning means designed to provide a user-recognisable engagement, e.g. by an ensuring “click”, the add-on device 200 is provided with a mounting switch 230, e.g. a mechanical micro switch, which is actuated from an off-state to an on-state when the add-on device is mounted on the pen housing.

FIG. 5 shows an add-on device 300 in a state where it has been mounted on the housing 101 of a drug delivery device of the above-described pen type. In contrast to the embodiment of FIG. 4 no user input button is provided. The device as shown is intended primarily to illustrate how an add-on device can be positioned on a pen device allowing a camera device (not shown) to capture images of the scale drum as presented in the housing display window 102. Correspondingly, portions of the add-on device have been removed.

The add-on device 300 as shown comprises a housing 301 with a cavity 305 having a lower opening adapted to be positioned over and in alignment with the housing display window 102. The opening is surrounded by a positioning structure in the form of a downwardly protruding lip portion 306 adapted to precisely engage and grip the chamfered edge portion 109 of the display opening, this ensuring that the add-on device can be correctly positioned on the pen housing. As will be explained in greater detail below the lip portion does not fully cover the edge portion surrounding the window opening. The add-on device further comprises a user-operatable locking member 360. The locking member may be designed to prevent locking until the add-on device is correctly positioned on the pen housing with the lip portion seated in the housing display opening. The mounting switch may be arranged to be actuated when the locking member is actuated to its fully closed position.

The above-described add-on device 200 is adapted to be mounted on a pen-formed drug delivery device of the type described above with reference to FIGS. 1-3, such a device comprising a scale drum with a plurality of dose size line markers as well as a window 102 with a pointer 109P. For a given set dose size the pointer will ideally be aligned with a line marker corresponding to that set dose size.

However, due to tolerances the scale drum may not be perfectly aligned rotationally with the pointer, which for a given set dose may result in the pointer not being perfectly aligned with the line marker for the actually set dose. For example, for a “true” set dose of 15 IU the scale drum may be positioned with the pointer arranged between 15 and 16 IU, i.e. at 15½ IU. Correspondingly, when the pointer points at ½ IU this may in fact represent 0 or 1 IU. Indeed, for small doses the relative inaccuracy may be quite significant.

For a typical drug delivery device each line marker on the scale drum is arranged with a rotational distance of 15 degrees, however, due the specific design of the expelling mechanism the distance between the “0” line marking and the “1” line marking may be smaller. For example, in the FlexTouch® pen device the distance between the arrow-formed “0” marking and the “1” line marking corresponds to a rotational distance of 9 degrees. For such a device the tolerances will most likely result in incorrect determination of the positions “0” and “1” due to the shorter distance between the two line markers, i.e. the pointer will point at the “½” position.

The present invention addresses the issue of finding the correct “0” (zero) position by determining an off-set value of the scale drum relative to the housing. This determination will of course be relevant for the determination of the correct zero position per se, however, as the entire scale drum will be rotationally off-set, the determination of a device specific scale drum rotational off-set may be used to correctly determine the rotational position of the scale drum for any given rotational position. This will be discussed in greater detail below.

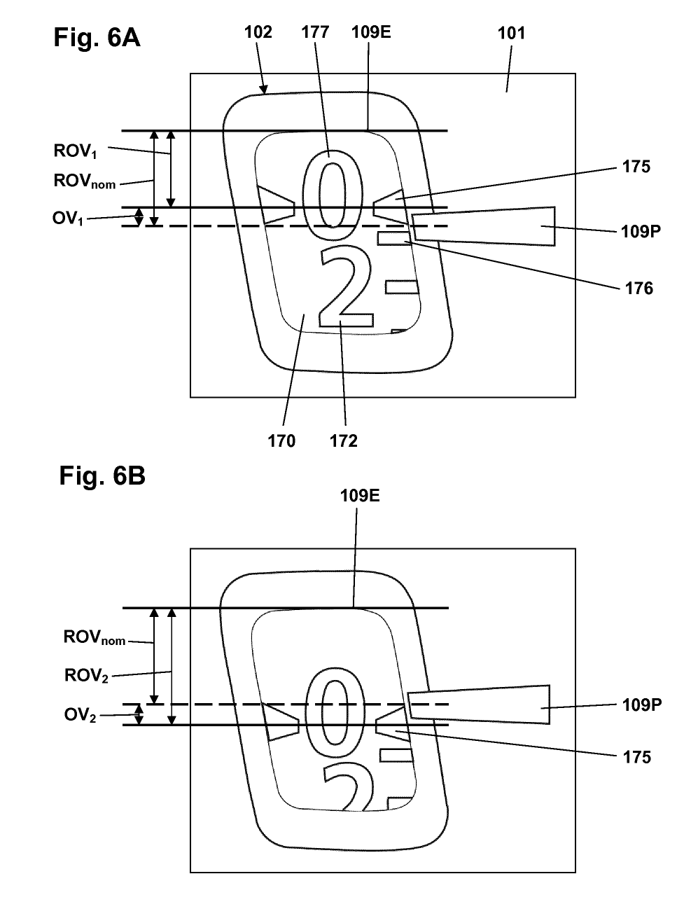

FIG. 6A shows a window portion of the drug delivery device described above with reference to FIGS. 1-3, the device comprising a drug expelling mechanism allowing a user to set a dose amount of drug to be expelled in increments of 1 IU. The drug delivery device comprises a scale drum indicator member 170 adapted to rotate relative to the housing 101 during dose setting and dose expelling corresponding to an axis of rotation, the amount of rotation corresponding to a set dose respectively the amount of drug remaining to be expelled from a reservoir by the expelling means, the indicator member having an initial rotational position corresponding to no dose amount being set. The housing 101 comprises an opening or display window 102 allowing a user to observe a portion of the scale drum indicator member 170, the opening being surrounded by a chamfered edge portion 109 and a dose pointer 109P. In the shown embodiment the axially oriented “upper” edge 109E (as seen in the figure) represents a “housing reference marker”. A pattern comprising a plurality of indicia is arranged helically on the indicator member. In the shown embodiment the indicia comprise a plurality of dose markers 176 as well as a plurality of associated numerals 172 comprising the equal numbers ranging from 0 to 80. The viewable dose marker positioned corresponding to the dose pointer 109P indicates to a user the currently set dose amount of drug to be expelled. The dose marker for 0 UI is in the form of a pair of opposed arrow markers whereas the remaining dose markers are in the form of a single line marker.

The scale drum is provided with an “initial pattern portion” observable by the user when the scale drum indicator member is positioned in the initial rotational position, the initial pattern portion comprising a “pattern reference marker”. In the shown embodiment the scale drum is provided with a “0” indicia 177, a 0 IU arrow marker 175 serving as the pattern reference marker, and a 1 IU line marker 176. In the shown embodiment the “initial pattern portion” is represented by a portion of the viewable scale drum comprising both the “0” indicia 177 and the arrow marker 175, e.g. the “upper half” of the viewable scale drum as seen in FIG. 6A. As the “0” used for the initial zero position is wider than the indicia “0” used in e.g. “10” or “20” it represents a unique marker.

In FIG. 6A the scale drum is arranged in the initial zero position, however, as can be seen the pointer 109P points to the portion of the scale drum between the 0 and the I IU markers, i.e. corresponding to a set dose of ½ IU. As dose amounts can only be set in increments of 1 IU the indication of dose size is incorrect, the correct dose size most likely being either 0 IU or 1 IU.

Addressing the issue of correctly determining the “true” initial position of a scale drum an exemplary add-on device, e.g. corresponding to the schematic representation in FIG. 4, is provided with a memory in which data representing the initial pattern portion is stored. The capturing means is adapted to capture an image of at least a portion of the initial pattern portion including the pattern reference marker as well as the housing reference marker. The processor is adapted to perform a reference offset value determination comprising the steps of (i) capture an image, (ii) perform an image analysis to determine if the captured image comprises the initial pattern portion, (iii) if the captured image comprises the initial pattern portion (indicating that the scale drum is in the initial position), then (iv) determine a “reference offset value” ROV₁ (see FIG. 6A) based on the distance between the housing reference marker (109E) and the pattern reference marker (175), the reference off-set value representing the initial rotational position; (v) if no reference offset value is stored in the memory or if the determined reference offset value corresponds to a smaller set dose amount than a currently stored value, then the determined reference offset value is stored in the memory.

To determine if the captured image comprises the initial pattern portion it may not be necessary to perform an image analysis of the entire captured image. For example, analysing only a portion of the captured image may be sufficient to identify the unique indicia “0” and thus the initial pattern portion. Indeed, the position of the pattern reference marker will also have to be determined.

As appears from the above, the stored reference offset value is dynamic and may be overwritten, the currently stored value representing the “true” initial position of the scale drum. The dynamic nature of the system can be illustrated by the following examples.

### Example 1

The add-on device is mounted on a new pen with no reference offset value stored. The dose has been set to “1” before the add-on device was mounted, however, the pointer points on “½” which is incorrectly interpreted as “0”, this corresponding to the situation in FIG. 6A.

### Example 2

The add-on device is mounted on a new pen with no reference offset value stored. The dose has been set to “20” before the add-on device was mounted, which means that initially no zero position will be identified and no offset value determined. When the set dose is expelled the scale drum normally returns to the initial zero position. However, the user may have stopped/paused the expelling action with 1 dose unit remaining. If the pointer points at “½” this may incorrectly be interpreted as “0”, which then will result in an incorrect dose being calculated (20 IU instead of 19 IU) as well as an incorrect reference offset value being stored. As for example 1, this situation corresponds to the situation in FIG. 6A.

However, due to the “dynamic” nature of the above-described concept the above two error conditions will most likely be corrected automatically as the above example 2 can be considered to be an “unusual” situation that will most likely rarely happen.

Correspondingly, when in example 1 an incorrect reference offset value has been stored initially, and the scale drum then subsequently at the end of an expelled dose returns to the true initial position, then the processor will identify the “0” indicia which will result in a reference offset value being calculated. However, as the value is not the same as the stored value, the stored value will be overwritten and a correct dose amount will be calculated.

This new initial position (which now is true) is shown in FIG. 6B in which the dose pointer 109P now points at “−½”. As the reference offset value ROV₂ is calculated as the distance between the upper window edge 109E and the arrow marker the new value is numerically larger than the previously determined value. If alternatively the reference offset value ROV₂ was calculated as the distance between the lower window edge and the arrow marker the new value would numerically be smaller than the previously determined value. In both cases the new determined reference offset value corresponds to a smaller set dose amount than the currently stored value.

In example 2 an incorrect reference offset value has been stored at the end of an expelling event and an incorrect dose size has been calculated. However, at the end of the next dose expelling event the scale drum will most likely return to the true initial position. The processor will identify the “0” marking which will result in a reference offset value being determined. However, as the value is not the same as the stored value, the stored value will be overwritten and a correct dose will be calculated. The fact that a new reference offset value has been determined may be utilized to correct a previously incorrectly determined dose size, e.g. in the above example 2 the stored log entry of 20 IU may be updated to 19 IU. Alternatively, in case log entries are stored in the form of captured data representing start and end rotational positions, a previously determined end position value may be updated, i.e. in example 2 from “0” to “1”.

The determined reference offset value may also be utilized to more safely and efficiently determine the rotational position of the scale drum indicator member, e.g. when the add-on device 400 scale drum position is determined by template-matching with a stored representation of the entire scale drum surface image.

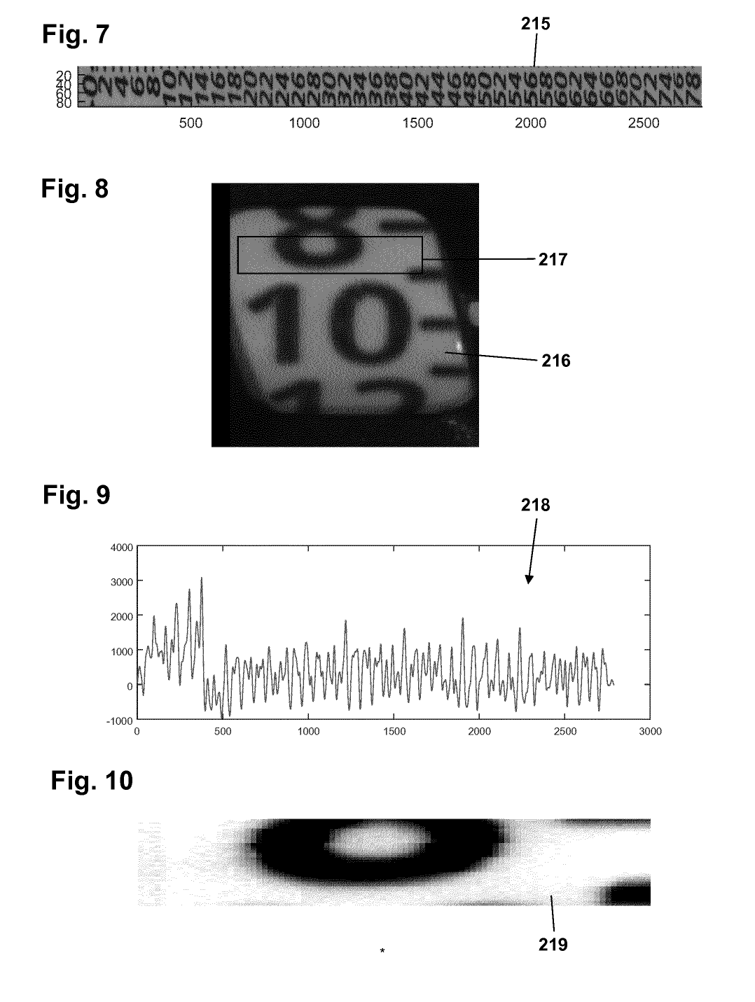

Correspondingly, FIG. 7 illustrates a template image 215 of the whole scale-drum. The image has been obtained by concatenating parts of successive images from a film where the scale-drum moves from position 80 to position 0. More specifically, the template has been made by concatenating the vertical centre of each frame in the movie, automatically creating a sheared image, this resulting in all digits being tilted as can be seen when compared to the scale drum digits shown in FIG. 8. Alternatively, the ribbon image could be obtained from a CAD-drawing of the scale drum print for a FlexTouch® device, the drawing being cut and sheared to produce a long ribbon. The template image is used as a reference when to determine the position of a specific image. The pixel position (horizontal axis in the above figure) corresponds to the drum position (in degrees, IU or other units). As an example, FIG. 8 shows an image 216 of the scale-drum window where the position corresponds to 10 IU, the rectangle 217 illustrating the area that is used for position detection. FIG. 9 then shows the cross correlation of the rectangle image portion to the reference 115 as a function 118 of pixel position. Searching for the peak reveals a best match at pixel position 341, corresponding to the cut 119 from the reference image as shown in FIG. 10. The reference image at this pixel offset was taken when the scale drum was in a position 9.8 IU. Indeed, if the template image has been created by sheering the digits this also means that the image taken with the camera should be sheered correspondingly before matching with the template.

In general, the captured image should be processed to correspond to the stored template, or, alternatively, the template image should be processed to correspond to the captured images before being stored. More specifically, in addition to the above-described shearing issue, the captured images may be distorted due to e.g. the angular orientation between the camera and the scale drum and the influence of any optical elements arranged in front of the camera. Correspondingly, the template image may be processed before storage to create a “distorted” image which matches the images as actually captured.

As appears, in case the scale drum indicator member is rotationally offset due to tolerances, a captured image for a given rotational position would not correctly correspond to the nominal template image for that rotational position. Correspondingly, if the scale drum rotational offset for a given drug delivery device could be determined, it would be possible to “shift” the template image to match the offset.

In the nominal initial rotational position the arrow marker 175 in FIG. 6A would be arranged exactly corresponding to the centre of the dose pointer 109P and thus have a nominal reference offset value ROVₙₒₘ. If for a given drug delivery device a reference offset value ROV₁ has been determined the ROVₙₒₘ could be used to calculate an actual offset value OV₁ as indicated in FIG. 6A, this value corresponding to the offset between an actual captured image portion and its corresponding nominal template image portion. In FIG. 6B a reference offset value ROV₂ has been determined and a corresponding offset value OV₂ has calculated.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. An add-on device configured to be releasably attached to a given drug delivery device, the drug delivery device comprising:
a drug reservoir or a compartment for receiving a drug reservoir,
drug expelling structure comprising a dose setting member allowing a user to set a dose amount of drug to be expelled,
an indicator member adapted to rotate relative to the housing during dose setting and dose expelling corresponding to an axis of rotation, the amount of rotation corresponding to a set dose respectively the amount of drug remaining to be expelled from a reservoir by the expelling structure, the indicator member having an initial rotational position corresponding to no dose amount being set,
a housing comprising an opening allowing a user to observe a portion of the indicator member, the opening being surrounded by an edge formed by the housing, the housing comprising a housing reference marker,
a pattern arranged circumferentially or helically on the indicator member, comprising:
a plurality of indicia, the currently observable indicia indicating to a user the size of a currently set dose amount of drug to be expelled, and
an initial pattern portion observable by the user when the indicator member is positioned in the initial rotational position, the initial pattern portion comprising a pattern reference marker,
the add-on device being adapted to determine, when mounted to a drug delivery device housing, an amount of rotation of the indicator member relative to the housing, the add-on device comprising:
mounting structure adapted to releasably mount the add-on device to
the drug delivery device in a predetermined position and orientation,
a memory,
capturing structure adapted to capture an image of:
at least a portion of the initial pattern portion including the pattern reference marker, and
the housing reference marker,
a processor adapted to (i) perform an image analysis to identify the initial pattern portion, and (ii) determine an amount of rotation of the indicator member relative to the housing based on input from the capturing structure,
wherein the add-on device is adapted to perform a reference offset value determination, comprising:
capture an image,
perform an image analysis to determine if the captured image comprises the initial pattern portion,
if the captured image comprises the initial pattern portion, determine a reference offset value (ROV) based on the distance between the housing reference marker and the pattern reference marker, the reference off-set value representing the initial rotational position,
(i) if no reference offset value is stored in the memory, store in the memory the determined reference offset value, or
(ii) if the determined reference offset value corresponds to a smaller set dose amount than a currently stored value in the memory, replace the currently stored reference offset value with the determined reference offset value.

2. An add-on device as in claim 1, wherein the housing reference marker is formed by a portion of the housing edge.

3. An add-on device as in claim 1, wherein the housing comprises a pointer structure and the plurality of indicia comprises a plurality of dose size markers, the pattern reference marker being formed by a dose size marker.

4. An add-on device as in claim 3, wherein the pattern reference marker is formed by the dose size marker for a set dose amount of zero.

5. An add-on device as in claim 1, wherein in the memory a reference representation of at least a portion of the initial pattern portion is stored, the processor being adapted to perform an image analysis comparing a captured image with the stored reference representation of at least a portion of the initial pattern portion.

6. An add-on device as in claim 1, wherein:
the pattern arranged on the indicator member forms a dosing pattern comprising a plurality of pattern portions, each pattern portion being arranged corresponding to a position on the indicator member,
a reference representation of the dosing pattern is stored in the memory, each part of the stored dosing pattern being correlated with a rotational position of the indicator member, and
the processor is adapted to perform a best-match analysis between a captured image and the stored reference representation to thereby determine the rotational position of the indicator member.

7. An add-on device as in claim 6, wherein:
for the given drug delivery device a nominal reference offset value (ROV-nom) is defined as the distance between the housing reference marker and the pattern reference marker in its nominal initial position, and
the add-on device is adapted to determine an offset value (OV) as the difference between the nominal reference offset value (ROV-nom) and a determined reference offset value (ROV).

8. An add-on device as in claim 7, wherein, before the best-match analysis is performed, the rotational correlation between a captured image and the stored reference representation is adjusted corresponding to a determined offset value.

9. An add-on device as in claim 6, wherein the capturing structure is arranged to capture an image with a given distortion, the reference representation being processed before storage to create a distorted representation matching the images actually captured.

10. An add-on device as in claim 6, wherein the capturing structure is arranged to capture an image with a given distortion, the captured image being processed before the best-match analysis to create an image matching the stored reference representation.

11. An add-on device as in claim 1, wherein an expelled dose amount is determined by the processor based on a first feature captured from a first image of the indicator member and a second feature captured from a second image of the indicator member, the first image being captured when a dose amount has been set and the second image being captured when a dose amount has been expelled.

12. An add-on device as in claim 11, wherein the first feature is used to determine a first position of the indicator member, and the second feature is used to determine a second position of the indicator member, the positional difference between the first and second positions being indicative of an expelled amount of drug.

13. An add-on device as in claim 1, wherein the processor is adapted to store in the memory data corresponding to one or more expelled dose amounts.

14. An add-on device as in claim 13, wherein stored data corresponding to one or more expelled dose amounts is updated when a stored reference offset value is replaced.

15. An add-on device as in claim 1, in combination with a drug delivery device as defined in claim 1.
